(19) United States
(12) Patent Application Publication
(10) Pub. No.: US 2008/0045627 A1
(43) Pub. Date: Feb. 21, 2008

(54) High Strength Bioreabsorbable Co-Polymers

(76) Inventor: John Rose, Collierville, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/565,029

(22) PCT Filed: Jul. 19, 2004
(86) PCT No.: PCT/GB04/03101

§ 371 Date: Sep. 11, 2006

(30) Foreign Application Priority Data
Jul. 19, 2003 (GB) 0317192.3

(51) Int. Cl. C08L 67/04; A61L 17/12; A61L 27/18; A61L 27/58

(52) U.S. Cl. 523/113; 264/328.1

(57) ABSTRACT

A polymer composition comprising poly-glycolic acid (PGA) and at least one other monomer to give a composition having a tensile strength of at least 1100 MPa.

The present invention relates to polymer compositions and artefacts made therefrom. In particular the present invention relates to polymers having high mechanical strength and their use for the manufacture of load bearing medical devices suitable for implantation within the body. More particularly the invention relates to bioresorbable glycolic acid-containing co-polymers and to implantable medical devices made therefrom.

Polymer compositions comprising poly-glycolic acid (PGA) and glycolic acid-containing copolymers have an established use for medical implants. It has also been proposed that certain mechanical properties may be improved by extruding PGA melts or by drawing PGA in a plastic state. Isotropic PGA has a tensile strength of between 50 to 100 MPa and a tensile modulus of between 2 and 4 GPa. A commercial product (SR-PGA) comprising PGA fibres in a PGA matrix has a flex strength and modulus of 200-250 MPa and 12-15 GPa, respectively. It is also reported in the literature that melt spun PGAs have tensile strength of about 750 MPa and a modulus from 15 to 20 GPa. In U.S. Pat. No. 4,968,317 an example of a drawn PGA is stated to have a tensile strength of about 600 MPa.

Although PGAs having improved strength characteristics are known, none of the known materials have the mechanical properties approaching those of the metals conventionally used for load bearing implantable medical devices. A commercial alloy used for orthopaedic implant devices, known as Ti-6-4, comprises titanium with 6% aluminium and 4% vanadium and has a tensile strength in the range of 800 to 1000 MPa and a modulus in the order of 100 GPa.

One possible reason that PGA and glycolic acid-containing co-polymers cannot currently be processed to achieve the desired strength of metals is that when the polymers are processed by common methods to produce orientated fibres (e.g. stretching the material at a constant rate in a heated chamber or tank) additional polymer crystallisation occurs during the process. The crystals in the polymer act such that they prevent further polymer orientation. This crystallisation of the polymer limits the mechanical properties that can be achieved by drawing glycolic acid-containing co-polymers to around 800 MPa, as described in the prior art.

We have found that polymer compositions comprising glycolic acid-based co-polymers may be processed such that the resultant composition has significantly greater strength, typically of the order of greater than 1100 MPa or 1150 MPa or 1200 MPa with a is commensurate increase in modulus, typically in excess of 20 GPa, 21 GPa or 22 GPa.

In accordance with the present invention there is provided a polymer composition comprising glycolic acid as a co-polymer with at least one other bioresorbable monomer, or a functional derivative of said co-polymer, having a tensile strength of at least 1200 MPa.

In accordance with the present invention there is provided a polymer composition comprising glycolic acid as a co-polymer with at least one other bioresorbable monomer, or a functional derivative of said co-polymer, having a tensile strength of at least 1100 MPa.

The polymer composition gains this level of tensile strength by means of a novel processing method that results in an orientated structure, for example an orientated fibre.

The present invention further provides an artefact comprising a polymer composition including glycolic acid or a functional derivative thereof having a tensile strength of at least 1200 MPa.

The present invention also provides an artefact comprising a polymer composition including glycolic acid or a functional derivative thereof having a tensile strength of at least 1100 MPa.

The polymer composition may be comprised entirely of glycolic acid-based co-polymer or a derivative thereof, or may comprise a glycolic acid-based co-polymer-containing blend with other polymers. Preferably the polymer composition is entirely glycolic acid-based co-polymer.

Similarly, artefacts formed from the polymer compositions of the invention may consist wholly of the polymer compositions of the invention or may be composites consisting only partially of the polymer compositions of the invention.

Aptly the artefact contains 10 to 80% by volume of the polymer compositions of the invention, suitably the artefact contains up to 60% by volume of the polymer compositions of the invention, preferably the artefact contains at least 40% by volume of the polymer compositions of the invention and typically the artefact contains approximately 50% by volume of the polymer compositions of the invention.

We have found that in order to achieve the high strength exhibited by the compositions of the invention it is necessary that the glycolic acid-containing co-polymer be rendered into an amorphous state and then immediately drawn to form a highly orientated structure.

This can be achieved by first processing isotropic glycolic acid-based co-polymer granules to form fibres or filaments, thereafter passing the fibres into a quenching bath to form an amorphous structure. Polymer compositions of the present invention may then be produced by drawing the quenched, amorphous glycolic acid based co-polymer. Preferably this is a drawing process which minimises the time polymer is exposed to elevated temperatures, thus minimising the time for the polymer to crystallise.

In accordance with another aspect of the invention there is provided a process for the manufacture of glycolic acid-based co-polymer compositions comprising increasing polymer chain orientation of a substantially amorphous polymer by drawing at localized points within the mass.

Suitably this comprises the steps of forming glycolic acid-based co-polymer or a functional derivative thereof into fibres, for example by melt extrusion or solution spinning; quenching the fibres then subjecting the quenched fibres to a tension under conditions whereby a defined region of the tensioned fibres is drawn.

Aptly fibres of amorphous glycolic acid-based co-polymer-containing polymers may be prepared by solution spinning or melt extruding the polymer through a die; the filament is then rapidly chilled to produce a substantially amorphous material. Typical chilling methods include blowing a cold gas onto the filament as it is produced or by passing the filament through a bath of a suitable cold liquid, e.g. water, silicone oil.

A suitable drawing method is zone heating. In this process a localised heater is moved along a length of fibre which is held under constant tension. This process is used in the zone-drawing process as described by Fakirov in Oriented Polymer Materials, S Fakirov, published by Hüthig & Wepf Verlag, Hüthig GmbH. In order to carry out this zone heating fibre can be passed through a brass cylinder. A small part of the cylinder inner wail is closer to the fibre, this small region locally heats the fibre, compared to the rest of the brass cylinder, localising the drawing of the fibre to this location, see FIG. 1. A band heater can be placed around the brass cylinder to allow it to be heated above room temperature. This heated brass cylinder can then be attached to the moving cross-head of a tensile testing machine and the fibre to be drawn suspended from a beam attached to the top of the testing machine. To draw the fibre a weight can be attached to the lower end of the fibre, the brass cylinder heated to the desired temperature and the cross-head moved to the lower end of the fibre, see FIG. 2. The polymer draws where the fibre is closest to the brass cylinder, as the cross-head is moved up the length of the fibre, then a length of the fibre can be drawn.

Suitably the fibre can be held taut using a small stress, which is typically below the yield point of the material at ambient temperatures. The fibre can then be heated locally to a temperature which is above the softening point (T_{g}) but below the melting point such that localised drawing of the polymer occurs, the whole fibre can be treated by movement of either or both the fibre and heated zone such that the full length of the fibre is drawn. This first drawing of the polymer may produce a polymer with improved molecular alignment and therefore strength and modulus. In this first step the conditions are selected such that the material does not substantially crystallise during the process, this requires that either the temperature of the polymer is below the temperature at which crystallisation occurs, T_{c}, or if the polymer is above T_{c} the speed at which the heated zone moves along the fibres is fast enough such that the polymer cools below T_{c} before it has time to crystallise. Further improvements can be made by subsequent treatments, where the stress applied to the fibre or the zone temperature is increased or both. Both the strength of the fibre and the softening point increase as the degree of molecular alignment improves. The process can be repeated many times, until the desired properties are reached. A final annealing step can be carried out in which the material crystallises under tension in the process; this can further improve the mechanical properties and improve the thermal stability of the final fibre.

In an embodiment of this aspect of the invention there is provided an artefact comprising a poly-glycolic acid in accordance with the invention. For example, the glycolic acid-containing co-polymer fibres can be mixed with other components to form the artefacts. These other components may be polymers, bioresorbable polymers, non-polymeric materials or combinations thereof.

Aptly the bioresorbable polymer comprises a poly-hydroxy acid, a poly-caprolactone, a polyacetal; a poly-anhydride or mixture thereof; the polymer comprises poly-propylene, poly-ethylene, poly-methyl methacrylate, epoxy resin or mixtures thereof whilst the non-polymeric component comprises a ceramic, hydroxyapatite, tricalcium phosphate, a bioactive factor or combinations thereof.

Suitably the bioactive factor comprises a natural or engineered protein, a ribonucleic acid, a deoxyribonucleic acid, a growth factor, a cytokine, an angiogenic factor or an antibody.

Artefacts according to the present invention can aptly be manufactured by placing appropriate lengths of strengthened glycolic acid-containing co-polymer fibre into moulds, adding the other components then compression moulding. Alternatively, the strengthened fibres can be pre-mixed with the other components then compression moulded.

In an alternative processing method, artefacts according to the present invention can be manufactured by forming a polymeric component in the presence of the strengthened fibres by in situ curing of monomers or other precursors for said polymeric component.

Preferably the monomers used in this process do not liberate any by-products on polymerisation as these can compromise the properties of the artefact.

Aptly at least one of the monomers used in said in situ curing process is a ring-opening monomer that opens to form a poly-hydroxy acid. Typically at least one monomer is a lactide, a glycolide, a caprolactone, a carbonate or a mixture thereof.

The polymer itself may be produced from reacting/incorporating/combining or by other means the glycolide or glycolic acid with at least one other monomer.

Incorporation of the at least one other monomer into the polymer composition can be achieved by any known means and for example maybe by ring polymerisation or transesterification.

Suitable monomers may include ring opening monomers like for instance lactide (& its isomers), trimethylene, carbonate, p-dioxanone, ε-caprolactone, 2-methyl glycolide, 2,3,2-dimethyl glycolide, 1,5-dioxapane, 1,4-dioxapane, 3,3-dimethyltrimethylene carbonate, glycosalicate, depsipeptides (morpholine 2,5-dione and related structures).

Aptly other suitable monomers may include Hydroxyacids, for instance including, lactic acid, caproic acid, hydroxyl benzoic acid and aminoacid esters.

In other embodiments the monomers may suitably be diacids (e.g. adipic acid, diglycolic acid), diols (e.g. propylene glycol, butane diol, or unsaturated diols like for instance hydroxyl propyl fumarates), addition monomers (e.g. spiro monomers, isocyanates, divinyl ethers), Anhydrides (e.g. sebacic anhydride).

The at least one other bioresorbable monomer component of the polymer composition according to the present invention may include a number of different monomers, in equal or different amounts.

Aptly the ratio of glycolic acid to bioresorbable monomer or monomers may be 95% PGA to 5% other monomer(s).

Typically the ratio of glycolic acid to other bioresorbable monomer/monomers will be 70:30%, 75:25%, 80:20%, 90:10%, 95:5% or 98:2%

Aptly there will be greater than 70% glycolic acid, in the polymer composition according to the present invention but aptly could also be greater than 75, 80, 90 or 95% glycolic acid to other bioresorable monomer/monomers.

Thus the bioresorbable monomer/monomers percentage may be aptly between 30 to 1%, 25 to 1%, 20 to 1%, 15 to 1%, 10 to 1% or 5 to 1%.

The polymer compositions of the invention are useful for the production of medical devices, particularly implantable devices where it is desirable or necessary that the implant is resorbed by the body. Thus, artefacts in accordance with the present invention include sutures; tissue-engineering scaffolds or scaffolds for implantation; orthopaedic implants; reinforcing agents for long fibre composites used in resorbable load bearing orthopaedic implants; complex shaped devices, for example formed by injection moulding or extruding composites formed by mixing short lengths of chopped fibres with poly-lactic acid; or bone fixation devices, for example formed from relatively large diameter rods (e.g., greater than 1 mm) of the compositions of the invention.

The invention will now be illustrated by the following example.

### EXAMPLE 1

PGA:PLA co-polymer (98% PGA, 2% PLA) was extruded into a water bath to produce a translucent fibre of approx 0.5 mm diameter. This fibre was then suspended vertically and a weight of 200 g was applied. A heated cylinder of brass with a hole of approx 15 mm apart from a small section with a 2 mm diameter hole, through which the PGA fibre passes, was heated to a temperature of 90° C. and moved along the fibre at a speed of 200 mm/min. The fibre produced was found to have a strength of greater than 1200 MPa and a modulus of greater than 20 GPa.

### EXAMPLE 2

A PGA-PLLA (poly-glycolic acid-poly L-lactide) (95:5%) co-polymer was extruded into a water bath to produce a translucent fibre of approximately 0.48 mm diameter. This fibre was then suspended vertically and a weight of 10 g was applied. A heated cylinder of brass with a hole of approximately 15 mm apart from a small section with a 2 mm diameter hole, through which the PGA fibre passes, was heated to a temperature of 90° C. and moved along the fibre at a speed of 500 mm/min.

The resultant fibre was tested in tension using an Instron 5566 machine fitted with a 100N load cell. Two pieces of the fibre were drawn and tested, the results are:

| | Strength/MPa | Modulus/GPa |
|---|---|---|
| Fibre 1 | 1154 | 21.4 |
| Fibre 2 | 1115 | 20.8 |

1. A polymer composition comprising glycolic acid (GA) as a copolymer with at least one other bioresorbable monomer, or a functional derivative of said co-polymer, having a tensile strength of at least 1100 MPa.

2. The polymer composition as claimed in claim 1, in which there are two bioresorbable monomers.

3. The polymer composition as claimed in claim 1, in which the at least one other bioresorbable monomer is polylactic acid (PLA).

4. The polymer composition as claimed in claim 1, in which the at least one other bioresorbable monomer is poly L-lactic acid (PLA).

5. The polymer composition as claimed in claim 1, in which the GA composition is at least 70% glycolic acid.

6. The polymer composition as claimed in claim 5 in, which the GA composition is at least 75, 80, 85, 90 or 95% glycolic acid.

7. The polymer composition as claimed in claim 4, in which the polymer composition is around 95% glycolic acid.

8. The polymer composition as claimed in claim 4, in which the polymer composition is around 98% glycolic acid

9. An artefact comprising strengthened glycolic acid polymer composition as claimed in claim 1.

10. The polymer composition as claimed in claim 1, in which the fibres have a tensile modulus of at least 20 GPa.

11. The polymer composition as claimed in claim 1, in which the fibres have a tensile modulus of at least 21 GPa.

12. The polymer composition as claimed claim 1, in which the fibres have a tensile modulus of at least 220 GPa.

13. A process for the manufacture of a polymer composition as claimed in claim 1, which includes the steps of:
a) forming the polymer composition comprising glycolic acid as a copolymer with at least one other bioresorbable monomer, or a functional derivative thereof, into fibre;
b) quenching the fibres; and
c) thereafter subjecting the quenched fibres to a tension under conditions whereby a defined region of the tensioned fibres is drawn.

14. The process according to claim 13, in which the fibre forming method is melt extrusion or solution spinning.

15. The process according to claim 13, in which the quenched, tensioned fibres are subjected to zone-heating.

16. The process according to claim 13, in which the quenched, tensioned fibres are subjected to at least two separate drawing steps, each drawing step performed under identical or different conditions.

17. An artefact comprising a polymer composition, or the functional derivative thereof according to claim 1.

18. The artefact of claim 17 comprising at least two polymer components.

19. The artefact of claim 18 comprising 10% to 80% by volume the polymer composition or the functional derivative.

20. The artefact of claim 17, in which at least one of the polymer components is bioresorbable.

21. The artefact of claim 20, in which the bioresorbable polymer comprises a poly-hydroxy acid, a poly-lactic acid, a poly-caprolactone, a poly-acetal or a poly-anhydride.

22. The artefact of claim 17 comprising at least one non-bioresorbable polymer component.

23. The artefact of claim 22 in which the non-bioresorbable polymer comprises poly-propylene, poly-ethylene, poly-methyl methacrylate or expoxy resin.

24. The artefact of claim 17 further containing at least one non-polymeric component.

25. The artefact of claim 24, in which the non-polymeric component comprises a ceramic, hydroxyapatite or tricalcium phosphate.

26. The artefact of claim 25, in which the non-polymeric component comprises a bioactive factor.

27. The artefact of claim 26, in which the bioactive component comprises a natural or engineered protein, a ribonucleic acid, a deoxyribonucleic acid, a growth factor, a cytokine, an angiogenic factor or an antibody.

28. The artefact according to claim 17, in which the artefact is in the form of a medical device.

29. The artefact of claim 28, in which the device is a suture, a scaffold for tissue engineering or implantation, an orthopaedics implant, a complex shaped device or a bone fixation device.

30. A process to manufacture of the artefact according to claim 17, comprising the steps of:
a) placing appropriate lengths of strengthened glycolic acid polymer composition comprising glycolic acid (GA) as the co-polymer with the at least one other bioresorbable monomer, or the functional derivative of said co-polymer, having the tensile strength of at least 1100 MPa into moulds;
b) adding and mixing any other components; and
c) compression moulding to the desired shape.

31. The process to manufacture of the artefact according to claim 17, comprising the steps of;
a) forming a polymeric component in the presence of strengthened glycolic acid polymer composition comprising glycolic acid (GA) as the co-polymer with the at least one other bioresorbable monomer, or the functional derivative of said co-polymer, having the tensile strength of at least 1100 MPa and;
b) in situ curing of the monomers or other precursors to form said polymeric component and artefact.

32. The process for the manufacture of the artefact according to claim 17, which includes the step of:
compression moulding other polymeric, non-polymeric or blend of polymeric and non-polymeric components in the presence of said fibres.

33. The process of claim 30, which further includes the step of:
compression moulding other polymeric, non-polymeric or blend of polymeric and non-polymeric components in the presence of said fibres.

34. The process of claim 32, which further includes the step of:
forming a polymeric component in the presence of said fibres by in situ curing of monomers or other precursors for said polymeric component.

35. The process of claim 34, in which the monomer used does not liberate a by-product on polymerisation.

36. The process of claim 34, in which at least one of the monomers is a ring opening monomer that opens to form a poly hydroxyl acid.

37. The process of claim 36, in which at least one monomer is a lactide, a glycolide, a caprolactone, a carbonate or mixtures thereof.

38. An artefact comprising a polymer composition, or the functional derivative thereof produced by the process according to claim 13.

39. The artefact of claim 38 comprising at least two polymer components.

40. The artefact of claim 39 comprising 10% to 80% by volume the polymer composition or the functional derivative thereof.

41. The artefact of claim 38, in which at least one of the polymer components is bioresorbable.

42. The artefact of claim 41, in which the bioresorbable polymer comprises a poly-hydroxy acid, a poly-lactic acid, a poly-caprolactone, a poly-acetal or a poly-anhydride.

43. The artefact of claim 38 comprising at least one non-bioresorbable polymer component.

44. The artefact of claim 43, in which the non-bioresorbable polymer comprises poly-propylene, poly-ethylene, poly-methyl methacrylate or expoxy resin.

45. The artefact of claim 38 further containing at least one non-polymeric component.

46. The artefact of claim 45, in which the non-polymeric component comprises a ceramic, hydroxyapatite or tricalcium phosphate.

47. The artefact of claim 46, in which the non-polymeric component comprises a bioactive factor.

48. The artefact of claim 47, in which the bioactive component comprises a natural or engineered protein, a ribonucleic acid, a deoxyribonucleic acid, a growth factor, a cytokine, an angiogenic factor or an antibody.

49. The artefact according to claim 38, in which the artefact is in the form of a medical device.

50. The artefact of claim 49, in which the device is a suture, a scaffold for tissue engineering or implantation, an orthopaedics implant, a complex shaped device or a bone fixation device.

51. A process to manufacture of the artefact according to claim 38, further comprising the steps of:
a) placing appropriate lengths of strengthened glycolic acid polymer composition comprising glycolic acid (GA) as the co-polymer with the at least one other bioresorbable monomer, or the functional derivative of said co-polymer, having the tensile strength of at least 1100 MPa, into moulds;
b) adding and mixing any other components; and
c) compression moulding to the desired shape.

52. The process to manufacture of the artefact according to claim 33, further comprising the steps of:
a) forming a polymeric component in the presence of strengthened glycolic acid polymer composition comprising glycolic acid (GA) as the co-polymer with the at least one other bioresorbable monomer, or the functional derivative of said co-polymer, having the tensile strength of at least 1100 MPa, and;
b) in situ curing of the monomers or other precursors to form said polymeric component and artefact.

53. The process for the manufacture of the artefact according to claim 38, which further includes the step of:
compression moulding other polymeric, non-polymeric or blend of polymeric and
non-polymeric components in the presence of said fibres.

54. The process of claim 51, which further includes the step of compression moulding other polymeric, non-polymeric or blend of polymeric and non-polymeric components in the presence of said fibres.

55. The process of claim 53, which further includes the step of:
forming a polymeric component in the presence of said fibres by in situ curing of monomers or other precursors for said polymeric component.

56. The process of claim 55, in which the monomer used does not liberate a by-product on polymerisation.

57. The process of claim 55, in which at least one of the monomers is a ring opening monomer that opens to form a poly hydroxyl acid.

58. The process of claim 57, in which at least one monomer is a lactide, a glycolide, a caprolactone, a carbonate or mixtures thereof.
